(19) United States
(12) Patent Application Publication
(10) Pub. No.: US 2018/0317900 A1
(43) Pub. Date: Nov. 8, 2018

(54) Atrial Retraction Device For Minimally Invasive Mitral Valve Procedures

(71) Applicant: University of Rochester, Rochester, NY (US)

(72) Inventors: Gregory Thomas Gdowski, Rochester, NY (US); Martin Gene Gira, Hilton, NY (US); Kyle Steven Meyers, Oneida, NY (US); Lauren Aimee Seitz, Deerfield, NH (US); Ian Benjamin Baranowski, Telford, PA (US); Margaret Rose Ferrari, North Dartmouth, MA (US); Fabio Molina Sagebin, Rochester, NY (US)

(73) Assignee: University of Rochester, Rochester, NY (US)

(21) Appl. No.: 15/970,675

(22) Filed: May 3, 2018

Related U.S. Application Data
(60) Provisional application No. 62/501,663, filed on May 4, 2017.

(51) Int. Cl. A61B 17/02; A61B 1/32

(52) U.S. Cl. CPC A61B 17/0218; A61B 1/00087; A61B 1/32

(57) ABSTRACT

An atrial retraction device is disclosed. In minimally invasive mitral valve surgery, there is often the need to make a second incision in the chest to place an atrial retractor for gaining clear access to the mitral valve of the heart by a cardiothoracic surgeon. The atrial retraction device of the present invention eliminates the need for a second incision, and provides an adjustable retraction device and associated surgical adjustor that reduces patient trauma, reduces surgical time, and does not require frequent adjustments during surgery. The atrial retraction device uses an adjustable band and roller arrangement to maintain clear and unobstructed access to the mitral valve during surgery.

### CROSS REFERENCE TO RELATED PATENT APPLICATIONS

This application claims priority to U.S. patent application Ser. No. 62/501,663 tiled May 4, 2017 entitled “Atrial Retraction Device For Minimally Invasive Mitral Valve Procedures”, the entire disclosure of which is incorporated herein by reference.

### BACKGROUND OF THE INVENTION

### 1. Field of the Invention

This invention relates generally to surgical assist devices, and more specifically to an atrial refraction device for minimally invasive mitral valve surgical procedures.

### 2. Description of Related Art

Mitral valve surgery, performed by cardiothoracic surgeons, is done to repair or replace the mitral valve as a result of valve regurgitation or leak, which if left untreated, can lead to heart failure and death. Two of the most common causes of mitral valve regurgitation are chordae tendineae ripping and valve prolapse. The chordae tendineae attach the leaflets of the mitral valve to the ventricle walls to allow the valve to properly open and close. Valve prolapse involves the valve leaflets becoming enlarged over time and eventually they are not able to open or close properly. This causes the leaflets to bulge into the atrium when the heart contracts and causes a misbalance of pressure. In both of these pathologies, the patient's effective blood ejection fraction (blood that exits to the body) is lowered which causes strain on their body as the amount of oxygenated blood that enters systemic circulation decreases. Furthermore, the heart attempts to compensate for the lack of blood by increasing its size (cardiac remodeling) to increase contractile force. This eventually leads to heart failure, as the cardiac tissue can no longer sustain repeated intense contraction.

Minimally invasive valve surgery is increasing in popularity due to its benefits to the patient when compared to the typical sternotomy approach. It eliminates the need to cut the sternum open and makes surgical recovery easier on the patient, as well as improving aesthetic value in regards to surgical scarring. The minimally invasive approach involves using a right thoracotomy, an incision between the ribs, as opposed to the sternotomy, which cuts through the breastbone and splits the chest in half. While the minimally invasive approach is usually better tor the patient, minimally invasive approaches leave the surgeon with less visibility of the valve he/she is working to repair. While there are currently devices used to improve visibility, they often require frequent adjustment throughout the procedure to be effective. This increases surgery time and are often difficult and inconvenient to use. The current devices in use today also require an additional incision in the chest near important arteries, which increase the risk of infection, bleeding, and patient recovery time.

Using the currently available devices, once the surgeon has made their thoracotomy incision, they would next make an additional incision into the top of the chest wall and insert a retractor rod. They then have to feed a footplate into the thoracotomy incision and line up the foot plate with the rod within the body. The retractor can then be adjusted and locked into place using an external clamp. This approach takes a great deal of time and has a large learning curve.

Currently available devices allow for only a certain amount of movement due to the restrictions from the chest incision and sometimes require the retractor to be redeployed through another new chest incision. In addition to this, the current retractors typically only provide retraction in one or two directions.

What is therefore needed is an atrial retraction device that eliminates a second or additional incision. What is farther needed is an atrial retraction device that deploys through the thoracotomy incision. What is also needed is an atrial retraction device that is simple and quick to adjust. What is further needed is an atrial retraction device that provides a surgeon with 360° of retraction, allowing the surgeon to see every part of the valve they are repairing or placing without obstruction.

### BRIEF SUMMARY OF THE INVENTION

In accordance with the present invention, there is provided an atrial retraction device for minimally invasive mitral valve surgery, the device comprising a device housing comprising a band guide and a roller guide; a baud having a terminated end and a free end, the terminated end being connected to the device housing; the band being configured to pass through the baud guide of the device housing; a roller having a shaft and a head; the shaft of the roller being retained in the roller guide of the device housing and frictionally engaged with the band such that rotation of the roller causes a change in diameter of the band.

The foregoing paragraph has been provided by way of introduction, and is not intended to limit the scope of the invention as described In this specification, claims, and the attached drawings.

### BRIEF DESCRIPTION OF THE DRAWINGS

The invention will be described by reference to the following drawings, in which like numerals refer to like elements, and in which;

FIG. 1 is a perspective view of an atrial retraction device of the present invention;

FIG. 2A is a perspective view of the atrial retraction device in a collapsed, non-deployed state;

FIG. 2B is a perspective view of the atrial retraction device in an expanded, deployed state;

FIG. 3 is a plan view of the atrial retraction device in a collapsed, non-deployed state;

FIG. 4 is a cross sectional view of the atrial retraction device taken along line A-A of FIG. 3;

FIG. 5 is a side plan view of the atrial retraction device;

FIG. 6 is an alternate plan view of the atrial retraction device in a collapsed, non-deployed state;

FIG. 7 is an alternate side plan view of the atrial retraction device;

FIG. 8 is a rotated side plan view of the atrial retraction device;

FIG. 9 is a further rotated side plan view of the atrial retraction device;

FIG. 10 is an exploded view of the atrial retraction device;

FIG. 11 is a perspective view of an adjustor coupled with the atrial retraction device;

FIG. 12 is a perspective view of an adjustor decoupled from the atrial retraction device;

FIG. 13 is a perspective view of another embodiment of an adjustor coupled with the atrial retraction device;

FIG. 14 is a perspective view of the adjustor of FIG. 13 decoupled from the atrial retraction device;

FIG. 15 is a perspective view of an adjustor and atrial retraction device with a quick release mechanism;

FIG. 16 is a photograph of an atrial incision (known as Waterston's groove) in a porcine heart used to access the mitral valve; and

FIG. 17 is a photograph of the atrial retraction device inside the left atrium of a porcine heart.

The present invention will be described in connection with a preferred embodiment, however, it will be understood that there is no intent to limit the invention to the embodiment described. On the contrary, the intent is to cover all alternatives, modifications, and equivalents as may be included within the spirit and scope of the invention as defined by this specification, claims and the attached drawings.

### DESCRIPTION OF THE PREFERRED EMBODIMENTS

For a general understanding of the present invention, reference is made to the drawings. In the drawings, like reference numerals have been used throughout to designate identical elements.

The present invention will be described by way of example, and not limitation. Modifications, improvements and additions to the invention described herein may be determined after reading this specification and viewing the accompanying drawings; such modifications, improvements, and additions being considered included in the spirit and broad scope of the present invention and its various embodiments described or envisioned herein.

Described and depicted herein is an atrial retraction device. Once a surgical incision is made into the left atrium to access the mitral valve, it is critically important to ensure that the cardiothoracic surgeon has unobstructed access to the mitral valve without interference from surrounding tissue. As has been previously described herein, a second incision is normally made through die chest to allow for insertion of an instrument that holds the atrium open. The present invention obviates the need for this second incision, and provides a device that expands in a circular manner to hold open the atrium during mitral valve repair and replacement surgeries.

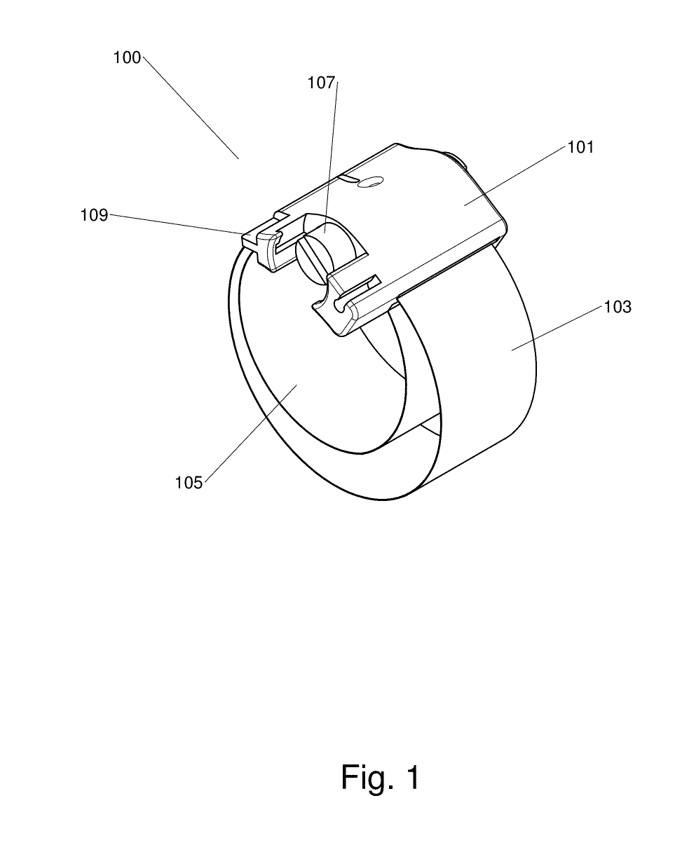

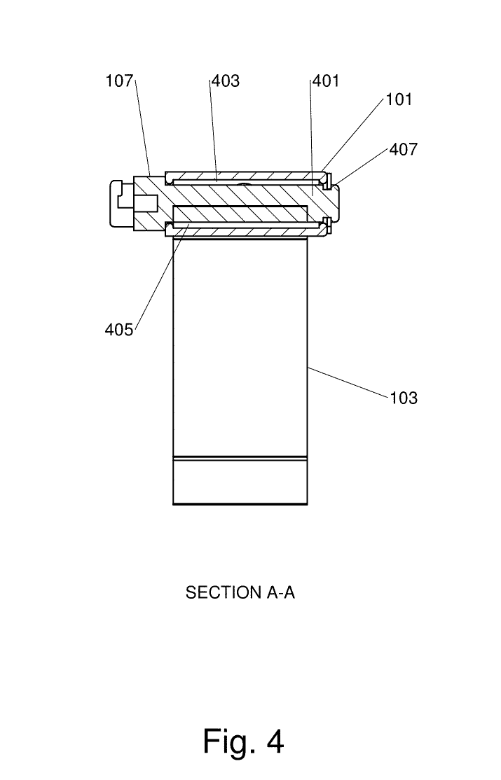

Turning first to FIG. 1, a perspective view of an atrial retraction device 100 of the present invention is shown. The atrial retraction device 100 for minimally invasive mitral valve surgery is deployed in a collapsed state, and with adjustment and rotation of a roller screw arrangement the diameter of the device increases, thus holding open the atrial incision to allow the surgeon unobstructed access to the mitral valve. There is a device housing 101 that retains a roller 107 and a band 103. The device housing 101 is made from a surgically compatible material such as various plastics, stainless steel, or the like, and has a band guide and a roller guide. The roller guide in one embodiment is a generally cylindrical opening in the device housing 101 that retains the roller 107. The band guide (see 403 and 405 in FIG. 4) is a slotted opening that retains the band 103 and allows the band 103 to pass by the roller 107 in such a way that the band 103 is in frictional contact with the roller 107.

The device housing 101 is circumferentially disposed with the band 103, and may, in some embodiments of the present invention, have a curved shape that conforms to the circular shape of the band 103, thus allowing unobstructed placement of the device within a patient. A protruding or otherwise incongruous device housing 101 would be problematic for placement by a surgeon. The head of the roller 107 may also be recessed or otherwise protected by the device housing 101 in some embodiments of the present invention. The device housing 101, as seen in the cross sectional view depicted in FIG. 4, has a first band guide 403 and a second band guide 405. The band guides are slotted or otherwise rectangular openings in the device housing 101 that allow for passage of the band 103 by the roller 107.

The band 103 is preferably made from a material that has spring-like properties, or in some embodiments of the present invention shape memory properties. The band 103 is expanded while under tension such that it has a natural affinity to collapse, allowing ease of diameter reduction and removal of the atrial retraction device 100 once the mitral valve procedure is complete. A surgically acceptable spring metal such as various grades and alloys of stainless steel or nitinol may be suitable for the band 103. In addition, while the band 103 is shown as a rolled up rectangular piece, it may vary in dimensions and may, in some embodiments, have additional geometries and features. The band 103 has a terminated end connected to the device housing 101 and a free end 105 that is taken up as the band circular structure is expanded during surgery. The free end 105 is rolled or coiled within the overall circular structure made by the band 103 so as not to interfere with vital surrounding heart tissue during surgery. The free end 105 is tensioned or formed in a way such that it adheres closely to the inner wail of the band circular structure and does not interfere with surgical procedures.

The band 103 is configured to pass through the band guide of the device housing 101. A roller 107 can be see having a shaft and a head. The roller shaft frictionally engages with the band 103 within the device housing 101 such that rotation of the head of the roller 107 with a suitable surgical tool allows the device to expand or contract in diameter. While the surgeon can adjust the diameter of the device to meet procedural and patient needs, various size atrial retraction devices may be provided to accommodate differing atrium sizes and anatomical conditions.

To facilitate removal, adjustment and placement of the atrial retraction device, a forceps retention surface 109 can be seen. This surface or structure may be a rectangular or otherwise protruding piece that can be grasped by a forceps or other suitable surgical tool. The forceps retention surface 109 may also be a recess or a slot in the device housing 101 or a protruding or extendable piece that is connected or formed with the device housing 101 in such a way that a surgical forceps or similar instrument can be used to grasp the atrial retraction device 100 and place, adjust or remove the device during a surgical procedure.

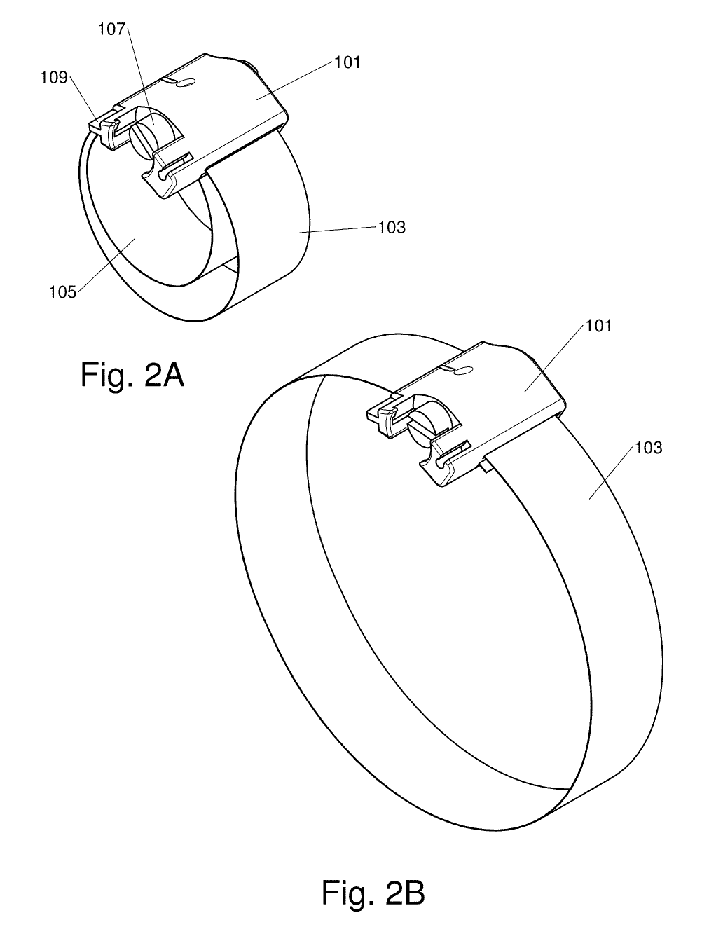

To better illustrate the atrial retraction device in both an expanded (deployed) and contracted (non-deployed) state. FIG. 2A is a perspective view of the atrial retraction device in a collapsed, non-deployed state and FIG. 2B is a perspective view of the atrial retraction device in an expanded, deployed state. It should be noted that the atrial retraction device may be adjusted within the range of fully collapsed to fully expanded to suit anatomical and surgical needs.

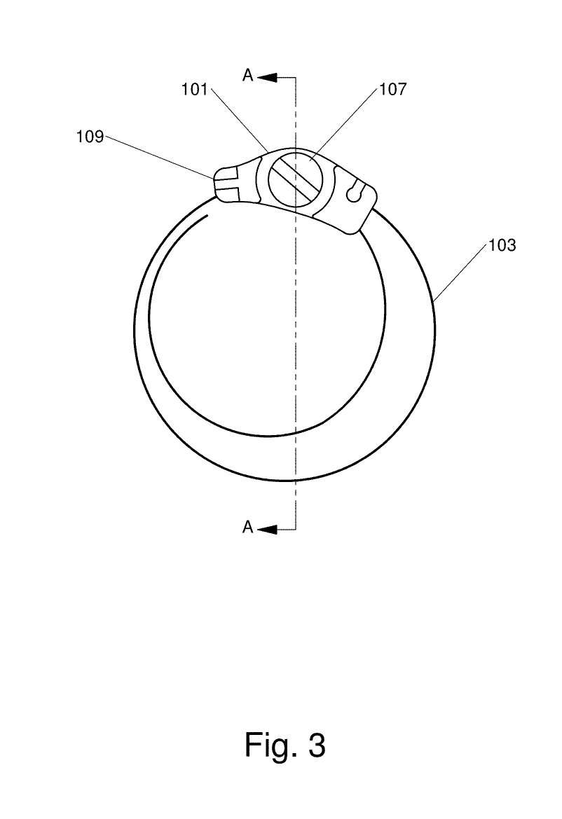

FIG. 3 is a plan view of the atrial retraction device in a collapsed, non-deployed state. While the free end of the band 103 is shown separated from the circular shape made by the band itself, in most embodiments the material characteristics of the band 103 will allow the free end to spring outward and conform to the inner wall of the formed circular band structure illustrated. Spring or shape retention materials allow for such outward deflection of the free end of the band.

FIG. 4 is a cross sectional view of the atrial retraction device taken along line A-A of FIG. 3. The roller 107 can be seen in the roller guide of the device housing 101. The roller 107 comprises a roller shaft 401 that may in some embodiments be coated, scored, textured, or otherwise have a friction enhancing material to ensure adequate transfer of torque from the roller 107 (being acted on by an external surgical tool) to the band 103 and further to provide for adequate shape retention of the band 103. An example of a friction enhancing material is a silicone rubber coating or covering. In some embodiments, the roller may have teeth or other features that engage with slots or holes in the band 103.

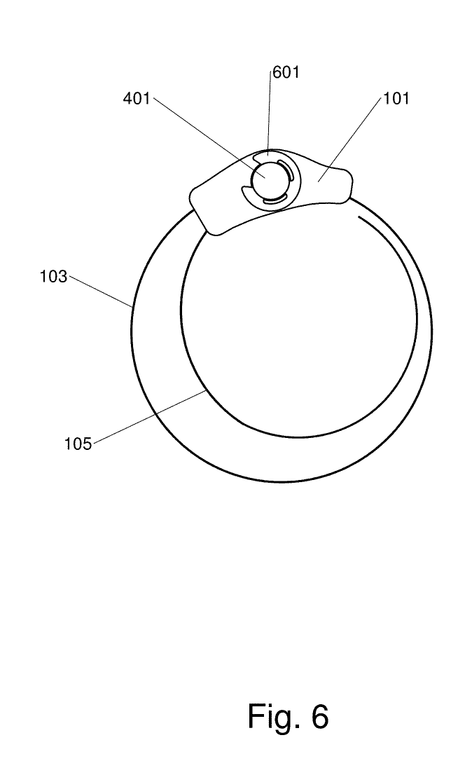

As described previously, a first band guide 403 and a second band guide 405 can be seen. While there are various techniques to retain the roller 107 within the roller guide, a circumferential retainer channel 407 can be seen with a retainer placed within (see FIG. 6, callout 601). While the roller head can be seen with a slot to receive a driver and impart rotational force to the roller in order to expand the device, other driver patterns may be employed such as a square drive, star drive, or the like.

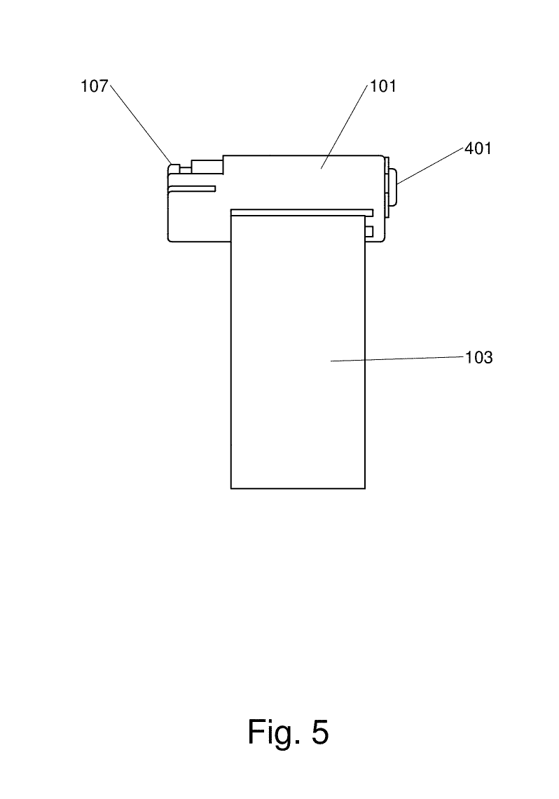

FIG. 5 is a side plan view of the atrial retraction device showing the relationship between the roller 107 and the band 103.

FIG. 6 is an alternate plan view of the atrial retraction device in a collapsed, non-deployed state. Again, the free end of the band is intentionally shown pulled away from the circular band structure in the interest of clarity. A retainer 601 can be seen in the retainer channel of the roller shaft 401. The retainer may be a metal or plastic clip, for example. Other retention techniques may also be employed, and are to be included in the present invention as described and envisioned herein.

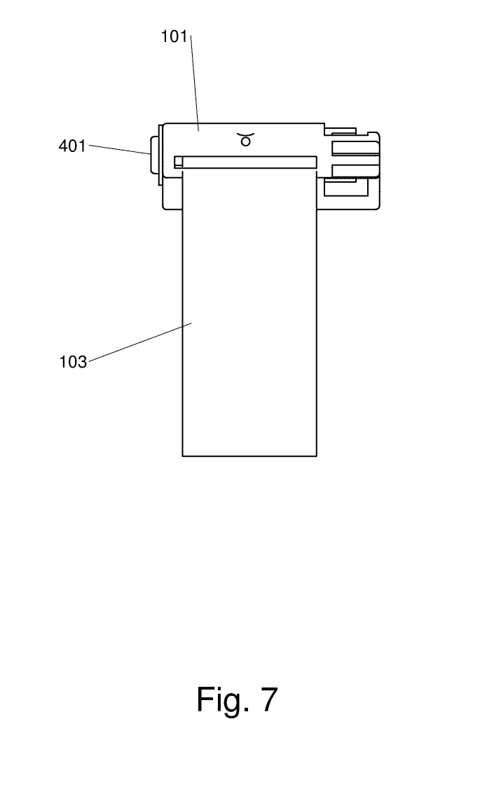

FIG. 7 is an alternate side plan view of the atrial retraction device showing the band 103 exiting the device housing 101. In some embodiments of the present invention, brakes, pins, teeth, or the like may be employed to increase the ability of the atrial retraction device to hold a set shape.

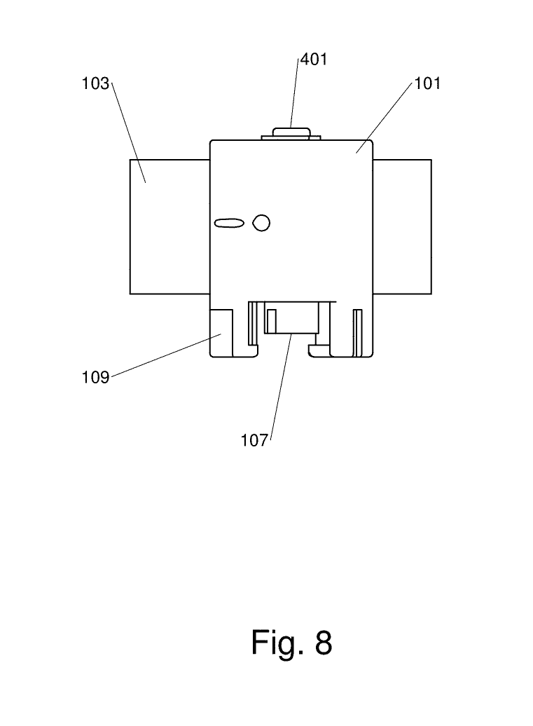

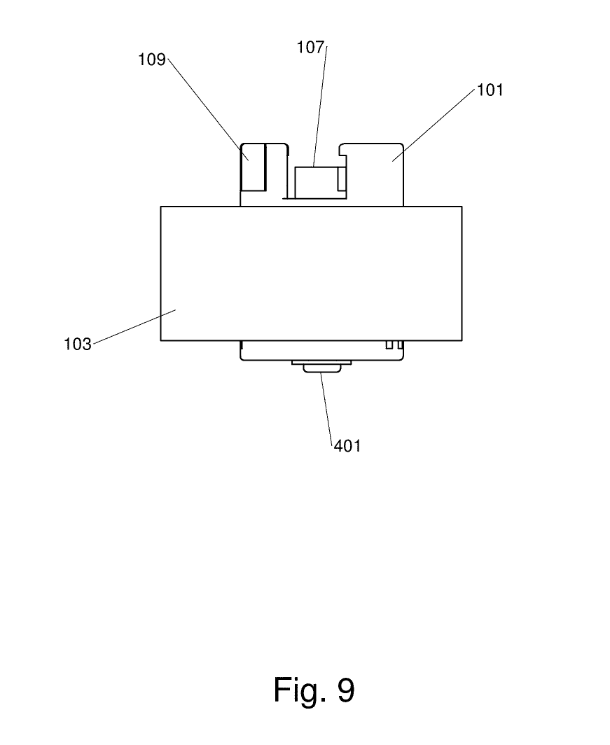

FIG. 8 is a rotated side plan of view of the atrial retraction device showing the roller head and insertion of the roller 107 through the device housing 101. FIG. 9 is a further rotated side plan view of the atrial retraction device.

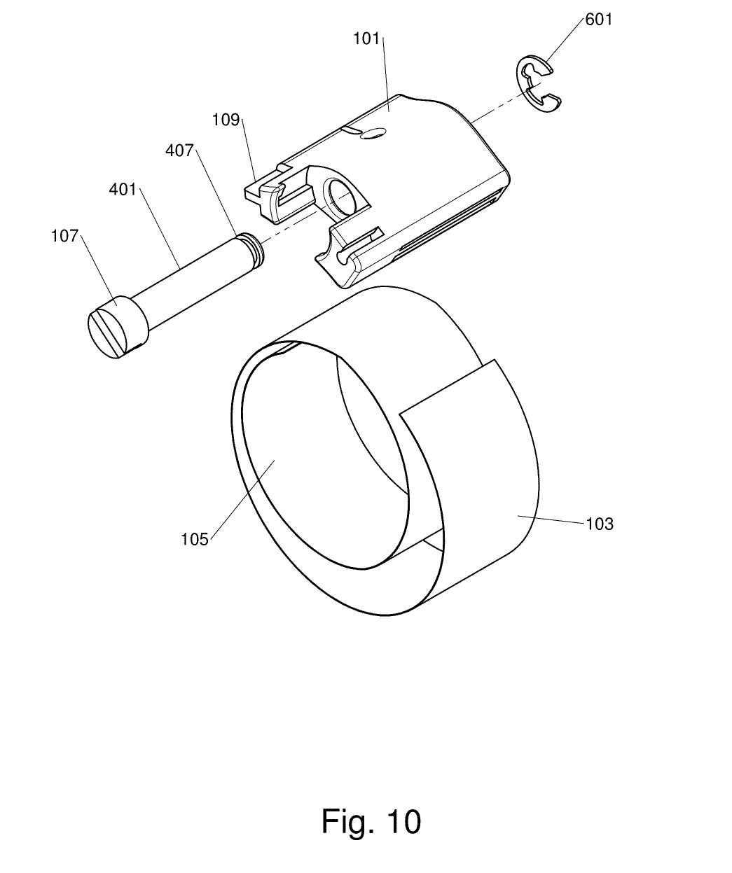

FIG. 10 is an exploded view of the atrial retraction device showing clearly the cooperation between the various components that have been heretofore described. The overall length of the band 103 may vary based on procedure, patient, surgeon preference, etc.

While the atrial retraction device may be adjusted with a suitable driver, the present invention includes an adjustor that engages with the atrial retraction device for ease of placement and adjustment. Several examples of such an adjustor are presented herein.

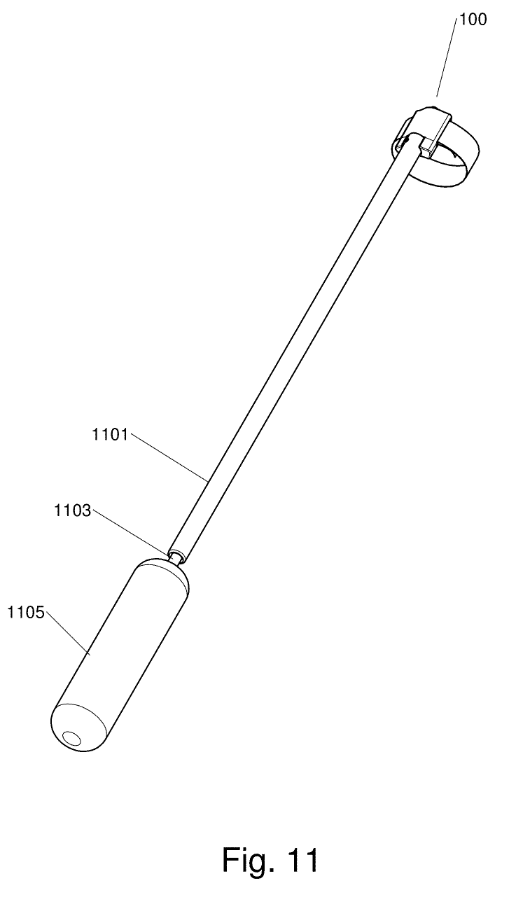

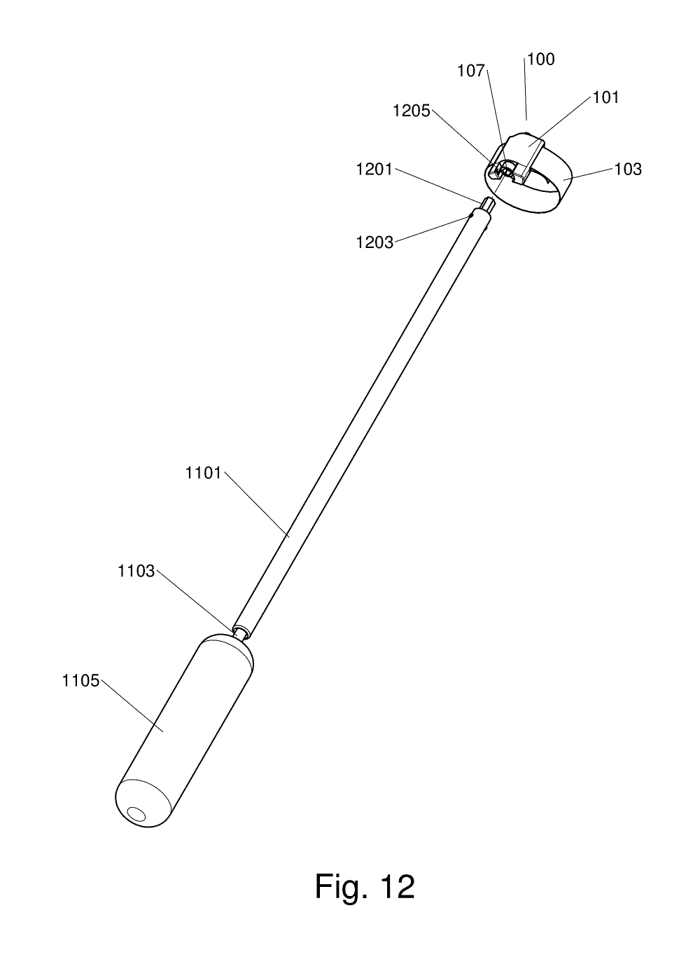

FIG. 11 is a perspective view of an adjustor coupled with the atrial retraction device 100. The adjustor comprises a shaft 1103 with a handle 1105 connected to a first end of the shaft and a coupling (see 1201, FIG. 12) connected to a second end of the shaft. The coupling has a driver pattern such as a slot, square drive, star drive, or the like and engages with the roller head of the atrial retraction device. Rotating the handle 1105 in turn rotates the roller 107 of the atrial retraction device to expand or collapse the device. A sleeve 1101 surrounds the shaft and serves not only as protection during surgery, but also engages with the device housing 101 of the atrial retraction device 100 to prevent rotation of the device during expansion or contraction of the band 103.

FIG. 12 is a perspective view of an adjustor decoupled from the atrial retraction device. The coupling 1201 can be seen that engages with the roller 107 of the atrial retraction device 100. An engaging member 1203 such as a protrusion or other such feature can be seen attached to the sleeve 1101 that engages with a receiving structure 1205 on the device housing 101. While there may be many variations of engaging members 1203 and receiving structures 1205, in the example depicted in FIG. 12, the protrusions internet with a slotted receiving structure 1205 to prevent rotation or movement of the atrial retraction device 100 during expansion or contraction of the band 103.

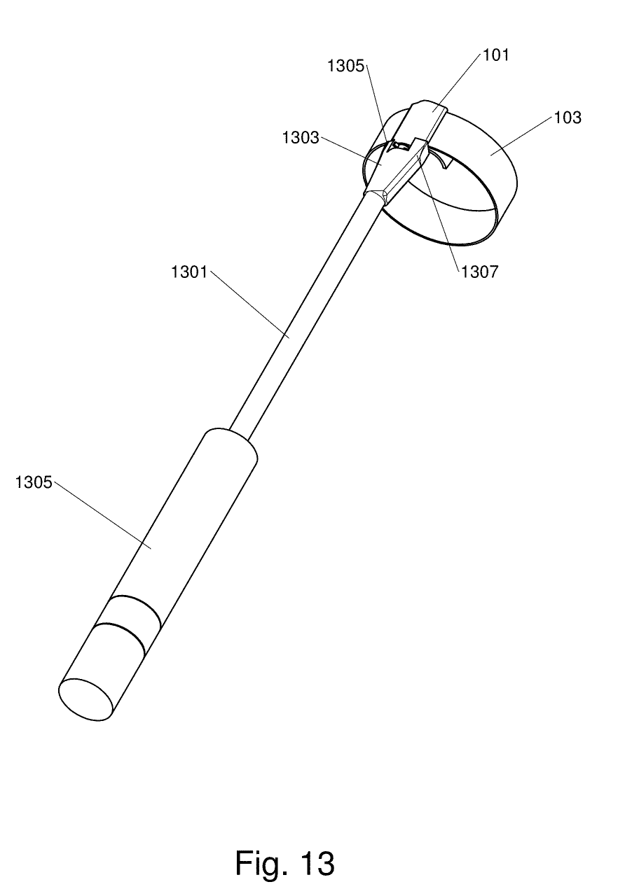

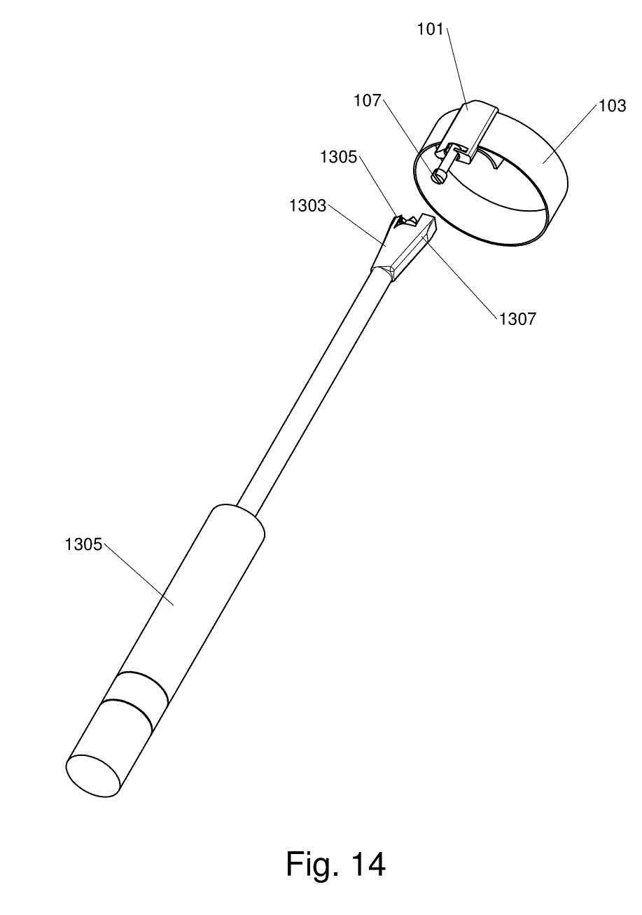

FIGS. 13 and 14 depict another embodiment of an adjustor. FIG. 13 is a perspective view of another embodiment of an adjustor coupled with the atrial retraction device. FIG. 14 is a perspective view of the adjustor of FIG. 13 decoupled from the atrial retraction device. The adjustor depicted comprises a shaft (not visible) that is covered by a sleeve 1301. An engaging member 1303 can be seen that has a first engaging element 1305 and a second engaging element 1307 that surrounds and interfaces with a receiving structure on the device housing 101. The first engaging element 1305 and the second engaging element 1307 can be seen as rectangular features or protrusions that originate from the engaging member 1303 and surround and mate with one end of the device housing 101. The engaging elements may have a channel slot or similar open portion that allows the device housing 101 to couple with the engaging member 1303 to prevent movement or rotation of the atrial retraction device 100 during placement or removal. It should be noted that preventing movement such as rotation of the atrial retraction device 100 is important, as such movement can create tissue trauma and damage, increase patient surgical time, and increase the probability of surgical error.

With an adjustor, the atrial retraction device 100 can be suitably deployed and sized. Once the mitral valve surgical procedure is complete, the atrial retraction device 100 can be collapsed through rotation of the roller 107 and extracted with either a forceps or the adjustor itself. In some embodiments of the present invention, the roller may be removable to facilitate the rapid collapse of the spring like band 103. If the roller 107 is removed either by a forceps or by way of an adjustor, it is important not to lose the roller 107, and attachment of the quick release roller 107 to the device itself through a tether or the like, is advantageous.

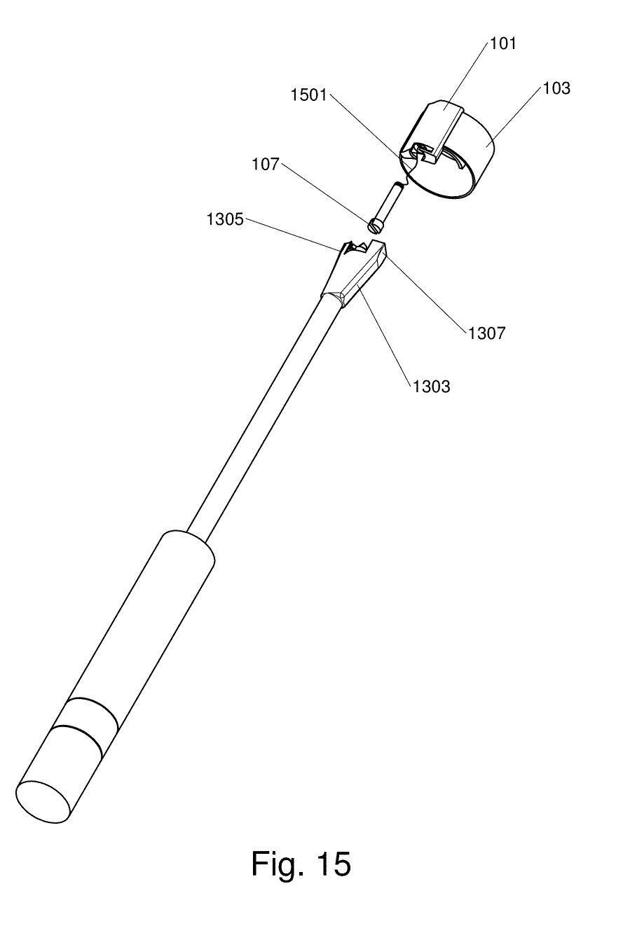

FIG. 15 is a perspective view of an adjustor and atrial retraction device with a quick release mechanism. Note that with a quick release mechanism where the roller 107 can be removed from the device housing 101, the retainer 601 as shown in FIG. 6 is either not used, or a retainer such as a protrusion, ridge, gasket, or the like is incorporated with the roller 107 in a way that allows the roller 107 to be removed with a given amount of force. For example, a gasket such as an O-ring, deformable skirt or other soft durometer material may be fastened to the roller shaft of the roller to provide frictional retention of the roller 107 within the device housing 101. Once the roller 107 is removed from the device housing 101, the spring like band will collapse, allowing rapid removal of the atrial retraction device from the patient and thus reducing surgical time. A tether 1501 can be seen connecting the roller 107 to the device housing 101. The tether 1501 can be a suitable surgically compatible string, cord, monofilament, or the like, and allows the roller 107 and the device to be removed as one piece with a forceps, adjustor, or other such surgical tool. The tether may also, in some embodiments of the present invention, be attached to the band 103, with suitable guide channels or holes in the device housing 101, such that when the roller 107 is pulled on and extracted, the tether 1501 serves to further collapse the band 103, facilitating size reduction and removal of the atrial retraction device.

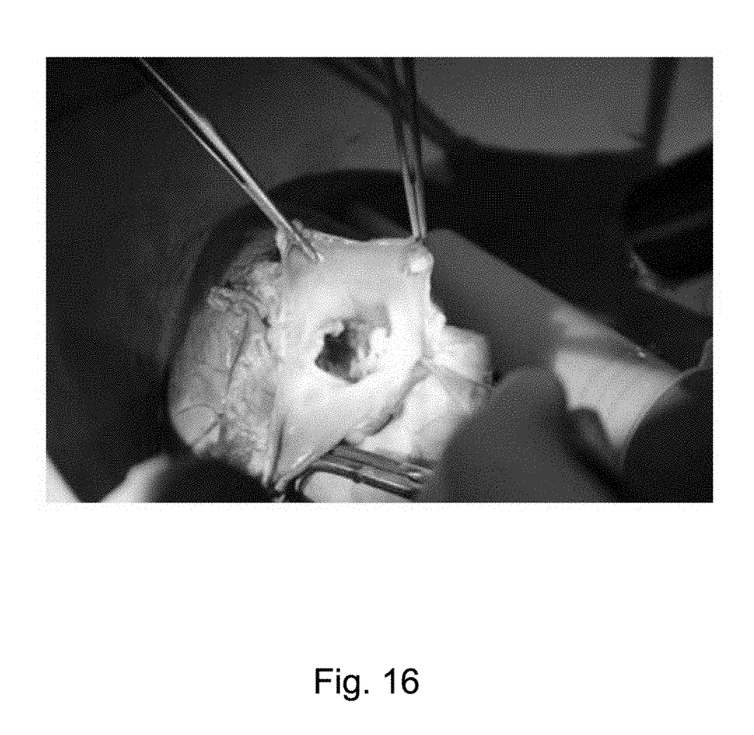

FIG. 16 is a photograph of an atrial incision (along Waterston's groove) in a porcine heart used to access the mitral valve, showing the necessity of a device to retract heart tissue during surgery.

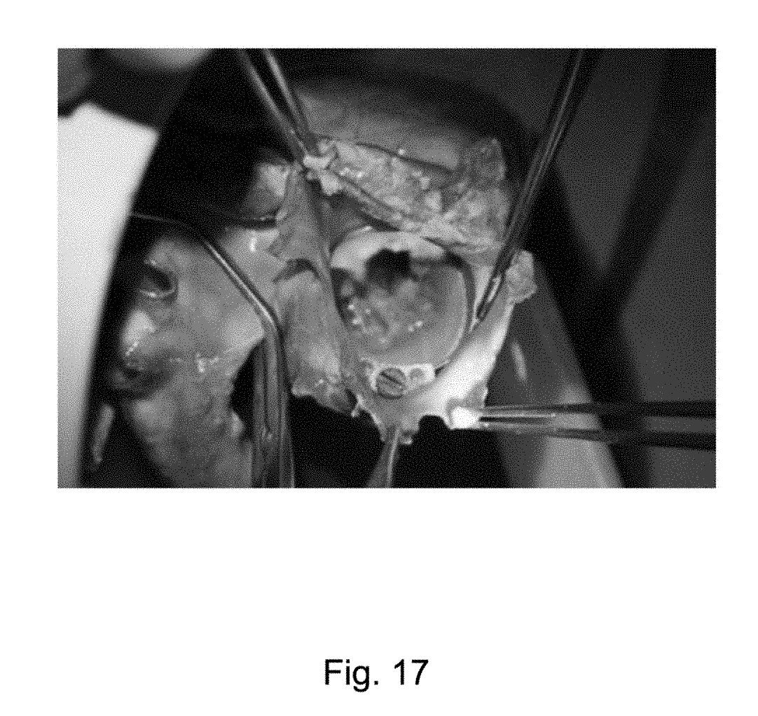

Lastly, FIG. 17 is a photograph of the atrial retraction device inside the left atrium of a porcine heart. The atrial retraction device has been expanded, showing clear access to the mitral valve.

While the various objects of this invention have been described in conjunction with preferred embodiments thereof, it is evident that many alternatives, modulations, and variations will be apparent to those skilled in the art. Accordingly, it is intended to embrace all such alternatives, modifications and variations that fall within the spirit and broad scope of this specification, claims and the attached drawings.

What is claimed is:

1. An atrial retraction device for minimally invasive mitral valve surgery, the device comprising:
a device housing comprising a band guide and a roller guide;
a band having a terminated end and a free end, the terminated end being connected to the device housing;
the band being configured to pass through the band guide of the device housing;
a roller having a shaft and a head;
the shaft of the roller being retained in the roller guide of the device housing and frictionally engaged with the band such that rotation of the roller causes a change in diameter of the band.

2. The atrial retraction device of claim 1, further comprising a friction enhancing coating on the roller shaft.

3. The atrial retraction device of claim 1, further comprising teeth on the roller shaft that are engageably connected with holes in the band.

4. The atrial retraction device of claim 1, wherein the band comprises nitinol.

5. The atrial retraction device of claim 1, wherein the free end of the band is retained by a circular loop made by the band.

6. The atrial retraction device of claim 1, further comprising a secondhand guide in the device housing.

7. The atrial retraction device of claim 1, further comprising a forceps retention surface on the device housing.

8. The atrial retraction device of claim 1, wherein the roller further comprises a retainer channel.

9. The atrial retraction device of claim 1, further comprising a retainer placed in the retainer channel for preventing the roller from leaving the device housing.

10. The atrial retraction device of claim 1, further comprising a tether between the device housing and the roller such that when the roller is removed from the device housing the band contracts in size and the device housing and the roller can be removed as one connected piece.

11. An atrial retraction device for minimally invasive mitral valve surgery, the device comprising:
a device housing comprising a band guide, a roller guide and a receiving structure;
a band having a terminated end and a free end, the terminated end being connected to the device housing;
the band being configured to pass through the band guide of the device housing;
a roller having a shaft and a head;
the shaft of the roller being retained in the roller guide of the device housing and frictionally engaged with the band such that rotation of the roller causes a change in diameter of the band;
an adjustor for placing and adjusting the atrial retraction device within the atrium of a patient, the adjustor comprising a shaft, a sleeve surrounding the shaft, a handle connected to a first end of the shaft and a coupling connected to a second end of the shaft.

12. The atrial retraction device of claim 11, wherein the adjustor further comprises an engaging member mechanically coupled to the sleeve of the adjustor and configured to engage with the receiving structure of the device housing of the atrial retraction device to prevent rotation and movement of the atrial retraction device during a surgical procedure.

13. The atrial retraction device of claim 12, wherein the engaging member of the adjustor further comprises a first engaging element and a second engaging element.

14. The atrial retraction device of claim 11, wherein the coupling of the adjustor is configured to connect with the head of the roller.

15. The atrial retraction device of claim 11, further comprising a friction enhancing coating on the roller shaft.

16. The atrial retraction device of claim 11, wherein the free end of the band is retained by a circular loop made by the band.

17. An adjustor for placing and adjusting an atrial retraction device within the atrium of a patient, the adjustor comprising:
a shaft having a first end and a second end;
a sleeve surrounding the shaft;
a handle connected to the first end of the shaft;
a coupling connected to the second end of the shaft; and
an engaging member mechanically coupled to the sleeve and configured to connect with an atrial retraction device to prevent rotation and movement of the atrial retraction device during a surgical procedure.

18. The adjustor of claim 17, wherein the engaging member of the adjustor further comprises a first engaging element and a second engaging element.

19. The adjustor of claim 17, wherein the coupling is configured to connect with a head of a roller of an atrial retraction device.

20. The adjustor of claim 17, wherein the coupling is configured to connect with and remove a roller of an atrial retraction device.
